(19) United States
(12) Patent Application Publication
(10) Pub. No.: US 2009/0117077 A1
(43) Pub. Date: May 7, 2009

(54) POLYETHYLENE GLYCOL-INTERFERON ALPHA CONJUGATE

(75) Inventors: Yeong-Woo Jo, Seoul (KR); Won-Young Yoo, Seoul (KR); Hyun-Kyu Jeon, Yongin-si (KR); Yun-Kyu Choi, Seongnam-si (KR); Hye-In Jang, Seoul (KR); Byong-Moon Kim, Seoul (KR); Sung-Hee Lee, Seoul (KR); Soo-Hyung Kang, Yongin-si (KR)

Correspondence Address: ROTHWELL, FIGG, ERNST & MANBECK, P.C., 1425 K STREET, N.W., SUITE 800, WASHINGTON, DC, 20005, US

(73) Assignee: DONG-A PHARM. CO., LTD., Seoul (KR)

(21) Appl. No.: 12/300,580

(22) PCT Filed: May 12, 2006
(86) PCT No.: PCT/KR06/01794

§ 371 Date: Jan. 22, 2009

(51) Int. Cl. A61K 38/21; C07K 19/00; A61P 35/02

(52) U.S. Cl. 424/85.7; 530/351

(57) ABSTRACT

The present invention relates to three-branched polyethylene glycol-interferon alpha conjugate of general formula (1) wherein polyethylene glycol has an average molecular weight of from 400 to 45,000 daltons, and a pharmaceutical composition comprising the same. The bioactive polyethylene glycol-interferon alpha conjugate of general formula (1) has antiviral and antitumoral activities, improved yield and purity by high reactivity in the reaction, and the effects to increase the half-life in blood remarkably, and to minimize the decreases in biological activity of interferon.

### TECHNICAL FIELD

The present invention relates to a three-branched polyethylene glycol-interferon alpha conjugate.

### BACKGROUND ART

Interferon was discovered in 1957 by Isaacs and Lindenmann, and has been known to have an excellent antivirus effect [Isaacs et al, Virus interference, 147 (1957)]. Interferon is classified into Type I (IFN-α, β, ω) and Type II (IFN- [symbol] ), and the cells generated by interferon are different such as white cell, fibroblast, T-cell, etc.

Modified interferon alpha was allowed and begun to be used as a therapeutic agent for hairy cell leukemia from 1986. Thus, interferon is the first cytokine produced by the gene recombination technology and used for treating cancer [Pestka et al, Semin. Oncol., 24 (1997)].

Interferon alpha is a pharmaceutically active protein having antiviral and antitumoral activities, and has been used for treating more than 14 classes of tumor and virus diseases in more than 40 nations in the world. Clinically effective treatment fields of interferon alpha are hairy cell leukemia, Kaposi's sarcoma, chronic Myelogenous Leukemia (CML), B-cell lymphoma, T-cell lymphoma, melanoma, myeloma, renal cell carcinoma [Nagabhushan T. L. et al, Regulatory practice for biopharmaceutical production, 221-234 (1994)].

Also, interferon is the first human protein that can increase the life span of cancer patient, and is expected to be able to be applied to different kinds of tumors such as ovarian cancer, breast cancer, bronchial cancer, bladder cancer, gastric cancer et al., and acute leukemia [Mosbe Talpaz et al, Seminars in Hepatology, 38(3), 22-27 (2001)].

Particularly, for treating type B or type C hepatitis, interferon alpha-2a (IFN alpha-2a), interferon alpha-2b, and interferon-con1 (IFN-con1) as mutein thereof are currently used. And, it has been reported that if an infection by virus such as type B hepatitis virus (HBV) or type C hepatitis virus (HCV) is chronically progressed, there is a risk that the infection can be progressed to hepatocellular carcinoma. Therefore, interferon can be used to prevent cancer.

However, interferon as clinically useful protein remedy has such problems as low stability in vivo, fast elimination in vivo, antibody formation by repeated administrations and hypersensitivity reaction thereby, like enzymes, proteins, hormones, peptides generated by genetic engineering method.

In particular, frequent administration such as once a day, 3 times a week, etc. induces pain to patients. Further, for those patients who need a treatment over a long period of time, such administration can threaten their quality of life.

To improve these problems, as a medicine that can be stable and maintain the activity over a long period of time, a protein remedy modifying polyethylene glycol was developed and has been currently used.

Polyethylene glycol is strongly hydrophilic, and can increase solubility at the time of bonding with therapeutic protein. Also, polyethylene glycol is effective for increasing the molecular amount of protein bonded thereto, with maintaining main biological functions such as enzyme activity and receptor binding. Thus, polyethylene glycol can decrease the glomerular filtration, and protect the protein effectively from proteolytic enzyme to decompose the protein. Therefore, polyethylene glycol has the advantages of preventing protein degradation, increasing the stability and circulation time of protein, and decreasing immunogenicity.

Commonly used linear polyethylene glycol has an molecular weight of about 1,000˜25,000 daltons, but it has a limitation in binding many linear high molecules to protein or peptide, with maintaining their activities, due to limited biological active regions of protein and peptide.

To improve these problems of linear polyethylene glycol, Wana, H et al tried to bind branched mono-methoxy polyethylene(mPEG) derivatives to protein by using trichlorotriazine [Wana, H et al., Ann. N.Y. Acad.Sci. 613:95-108 (1990)].

However, the size of activated branched polyethylene glycol derivatives is big, and so induces steric hinderance on the surfaces of protein or peptide, thereby reducing the activities of modified protein or peptide. Also, the derivatives usually cause low yield of purification due to incomplete branched polyethylene glycol derivatives.

Korean Patent No. 0396983 tried to improve these problems of the branched high molecular derivatives. In particular, the patent tried to minimize the reduction of biological activity by protecting the protein structure through minimizing the number of linkers bonded to biologically active regions by lengthening the linkers to connect high molecule and protein, and reducing steric hindrance induced by the branched high molecules. However, tri-PEG-NHS that is activated branched high molecular derivatives having long linkers contains excess linear PEG-NHS and Di-PEG-NHS having small molecular amounts as impurities when the linker structure is prepared. They competitively participate in the bonding reaction to interferon, and generate low molecular PEG-interferon alpha conjugate and Di-PEG-interferon alpha conjugate which are difficult to purify. Thus, this method has low purity and low yield problems.

Therefore, there still has been a need for macromolecular polyethylene glycol-interferon conjugate that can minimize reduction of the bioactivity of interferon alpha, and have high purity and good stability.

### DISCLOSURE OF INVENTION

### Objections of the Invention

The object of the present invention is to provide three-branched polyethylene glycol-interferon alpha conjugates, having high production purity and yield, increasing the half-life in blood, and minimizing the bioactivity reduction of interferon in comparison to interferon alpha and polyethylene glycol-interferon alpha conjugates known in the art; a preparation method of the same, and a pharmaceutical composition containing the same.

### Technical Solution

To achieve the above object, the present invention provides a high molecule binding three-branched polyethylene glycol derivatives to interferon alpha, and having high purity, and a pharmaceutical composition containing the molecule.

The present invention is explained in detail below.

Polyethylene glycol-interferon alpha conjugate is generated by a binding reaction of three-branched polyethylene glycol derivatives and interferon alpha, and can be represented by the following general formula (1),

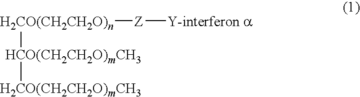

wherein n is an integer of 1 to 1,000, and m is an integer of 10 to 1,000.

In the above conjugate, the average molecular weight of polyethylene glycol is from 400 to 45,000 daltons, preferably 30,000 to 45,000 daltons, more preferably 43,000 daltons.

As the molecular weight of polyethylene glycol is higher, the pharmacokinetics of high molecular conjugate is better, but the activity is decreased. So, a proper molecular weight is important.

Z is (CH₂)_{S} or (CH₂)_{S}NHCO(CH₂)_{S} to play a linker role of interferon alpha and polyethylene glycol, wherein S is an integer of 1 to 6. Y is a secondary amine or an amide bond, formed by a bonding reaction of NH₂ functional group of interferon molecule and a functional group of polyethylene glycol derivative.

Also, the present invention provides a method of preparing three-branched polyethylene glycol-interferon alpha conjugate as shown in the following general formula (1) wherein polyethylene glycol has an average molecular weight of from 400 to 45,000 daltons, preferably 30,000 to 45,000 daltons, more preferably 43,000 daltons.

Three-branched polyethylene glycol derivatives of the present invention are activated high molecule having branched structure that three linear biological receptive high molecules are combined. All of three OH (hydroxy) regions in the glycerol structure are polymerized with ethylene glycol unit molecules, and the end of one region is activated as a functional group. The other two regions except the activated region are substituted with monomethoxy to prevent additional reactions. When the above branched polyethylene glycol derivatives are prepared, the size of each linear polyethylene glycol can be controlled freely, whereby a high molecule having proper structure and molecular weight can be prepared and bonded to interferon alpha.

A branched polyethylene glycol (PEG) derivative bonding to interferon alpha is represented by the following general formula (2)

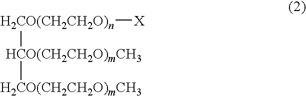

wherein, n is an integer of 1 to 1,000 and m is an integer of 10 to 1,000. The average molecular weight of polyethylene glycol unit of the conjugate is from 400 to 45,000 daltons, preferably 30,000 to 45,000 daltons, more preferably 43,000 daltons. X is a functional group that can react chemically to protein or peptide containing interferon alpha, as shown in the general formula (3) below. Preferably, X is N-hydroxysuccin imide (a) or aldehyde (b) in the compound of formula (3), and each forms amide bond and secondary amine structure bond in the bonding reaction to interferon alpha in high yields.

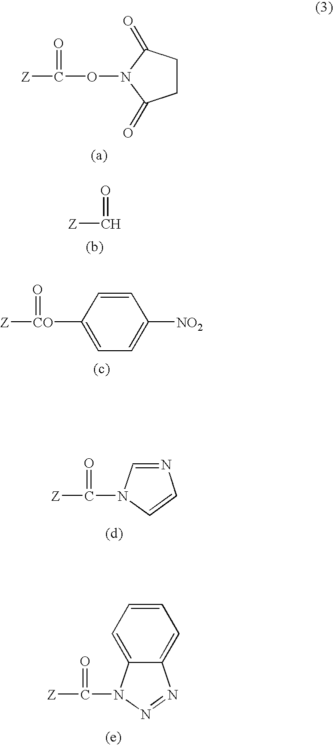

Z is (CH₂)_{S} or (CH₂)_{S}NHCO(CH₂)_{S} to play a linker role of interferon alpha and polyethylene glycol wherein S is an integer of 1 to 6.

In this invention, the reaction molar ratio of interferon alpha to the branched polyethylene glycol derivative is from 1:0.5 to 1:50. Preferably, the molar ratio of interferon alpha to the branched polyethylene glycol derivative is from 1:0.5 to 1:3. As the molar ratio of polyethylene glycol to interferon alpha is increased, the yield of mono polyethylene glycol-interferon alpha conjugate per unit time is decreased.

Also, the present invention provides a pharmaceutical composition for treating or preventing interferon alpha receptive diseases, comprising a polyethylene glycol-interferon alpha conjugate according to this invention as an effective ingredient. The composition can be composed of an effective dose of polyethylene glycol interferon alpha conjugate of the present invention, diluent, antiseptics, solubilizer, emulsifier, juvantia, and/or carrier.

The pharmaceutical compositions of the present invention can be formulated to an injection agent, a capsule, a tablet, a liquid drug, a pill, an ointment, an oculentum, a collyrium, a transdermal absorptive agent, a paste, a cataplasm, a patch agent, an aerosols, etc. And, the effective dosage of pharmaceutical composition of the present invention may be varied according to patient's age, condition, weight etc, but generally once a week or once two weeks. And, the composition can be administrated once or many times a day within a daily effective dosage range.

Further, the present invention provides a method of treating or preventing interferon alpha receptive diseases, comprising administering the conjugate of the present invention as an effective ingredient. The interferon alpha receptive diseases include hairy cell leukemia, Kaposi's sarcoma, chronic Myelogenous Leukemia(CML), B-cell lymphoma, T-cell lymphoma, melanoma, myeloma, renal cell carcinoma. And the disease includes ovarian cancer, breast cancer, bronchial cancer, bladder cancer, gastric cancer, etc and the other cancers like acute leukemia.

The present invention is explained particularly by the following examples. The following examples are intended to further illustrate the present invention, but the scope of the present invention is not intended to be limited thereby in any way.

### EFFECTS OF THE INVENTION

The present invention relates to biologically active new three-branched polyethylene glycol-interferon alpha conjugates having glycerol structure. So, this invention is characterized in having high purity and high yield, minimizing the reduction of bioactivity, and increasing the half-life in blood, by overcoming the problems that linear polyethylene glycol cannot bond many linear high molecules to protein or peptide; branched high molecular derivatives induce excessive steric hindrance on the surfaces of protein or peptide; and branched high molecular derivatives whose linkers are lengthened have low purification yield by low purity, etc.

Therefore, the pharmaceutical composition of the present invention containing polyethylene glycol-interferon alpha conjugate having antivirus activity and anti-tumor activity has the effects that the reduction of activity is minimized, and the treatment effect can be improved, and the patient's uncomfortableness can be minimized by decreasing the administration frequency due to the lengthened half-life in body, compared to interferon alpha treatment agent known in the art.

### DESCRIPTION OF THE DRAWINGS

FIG. 1 is a schematic drawing illustrating the analytic results of Example 1 by size-exclusion high performance liquid chromatography(hereinafter: SE-HPLC).

FIG. 2 is a schematic drawing illustrating the analytic results of Example 2 by SE-HPLC.

FIG. 3 is a schematic drawing illustrating the analytic results of Comparative Example 1 by SE-HPLC.

FIG. 4 is a schematic drawing illustrating the analytic results of Comparative Example 2 by SE-HPLC.

FIG. 5 is a schematic drawing illustrating the analytic results of Comparative Example 3 by SE-HPLC.

FIG. 6 is a schematic drawing illustrating the analytic results of Comparative Example 4 by SE-HPLC.

FIG. 7 is a schematic drawing illustrating the analytic results of Example 1 by Matrix-Assisted Laser Desorption Ionization—Time Of Flight (MALDI-TOF) Mass Spectrometer(hereinafter: MALDI-TOF).

FIG. 8 is a schematic drawing illustrating the analytic results of Example 2 by MALDI-TOF.

FIG. 9 is a schematic drawing illustrating the analytic results of Comparative Example 1 by MALDI-TOF.

FIG. 10 is a schematic drawing illustrating the analytic results of Comparative Example 2 by MALDI-TOF.

FIG. 11 is a schematic drawing illustrating the suppression results of the cytopathic effects (CPE) of interferon alpha conjugates modified with polyethylene glycol of Example 1, and Comparative Examples 1 and 3 by using vesicular stomatitis virus and Marbin-Darby Bovine Kidney cells (MDBK).

FIG. 12 is a schematic drawing illustrating the comparative analytic results of pharmacokinetics of interferon alpha and the polyethylene glycol-interferon alpha conjugate of Example 1.

FIG. 13 is a schematic drawing illustrating the effect comparison results of antitumor activities of interferon alpha, and the polyethylene glycol-interferon alpha conjugate of Example 1 by using Daudi cells.

FIG. 14 is a schematic drawing illustrating the comparison results of biological activity changes of interferon alpha, and interferon alpha conjugate modified with three branched polyethylene glycol (PEG, MW43,000)—of Example 1 according to temperature change.

FIG. 15 is a schematic drawing illustrating the analytic results of biological activities of interferon alpha, and interferon alpha conjugates modified with the polyethylene glycol of Example 1 to proteolytic enzyme and time.

### MODE FOR THE INVENTION

### Example 1

### Preparation of Three-branched Polyethylene Glycol (MW 43,000 Da)-interferon Alpha Conjugate (I) of the Formula (1) By Using Three-branched Polyethylene Glycol N-hydroxysuccinimide

68 mg of three-branched polyethylene glycol N-hydroxysuccinmide (NOF corporation, Japan) having the molecular weight of 43,000 daltons were added to 10 mg of interferon alpha (Dong-A Pharm. Co., Ltd.) in 100 mM of bicine buffer, pH8.0. The reaction mixture was stirred for 2hr at room temperature. And, the reaction was stopped by adding 0.1M of glycine.

The reactant was inputted into Hiprep™ 26/10 (Amersham Pharmacia Biotech) desalting column equilibrated with 40 mM of NaH₂PO₄ (pH4.0) buffer solution and the buffer solution was changed by eluting with same buffer solution. N-hydroxysuccinimide separated from the three-branched polyethylene glycol-N-hydroxy succinimide by this reaction was removed. An eluant was inputted into SP-Sepharose Fast Flow cation exchange chromatography (Amersham Pharmacia Biotech) equilibrated with 40 mM of NaH₂PO₄ (pH4.0) buffer solution, and then polyethylene glycol-interferon alpha conjugate was separated by the liquid chromatography.

The polyethylene glycol-Interferon alpha conjugate was fractioned using 0˜500 mM of concentration gradient of sodium chloride(NaCl).

The form and size of the fractioned eluate was confirmed by HPLC and SDS-PAGE. And the conjugates of forms bonded di- or tri-three-branched polyethylene glycols with an interferon alpha, and unmodified interferon alphas remaining after the reaction were excluded, to obtain the title conjugate, polyethylene glycol interferon alpha conjugate bonded one three-branched polyethylene glycol (MW 43,000) with an interferon alpha(or called as mono-three-branched polyethylene glycol-interferon alpha conjugate).

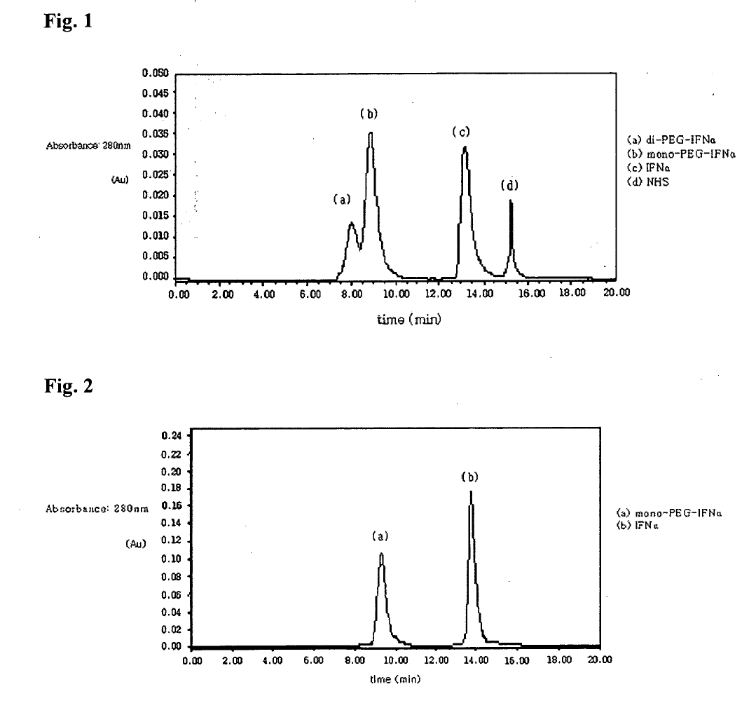

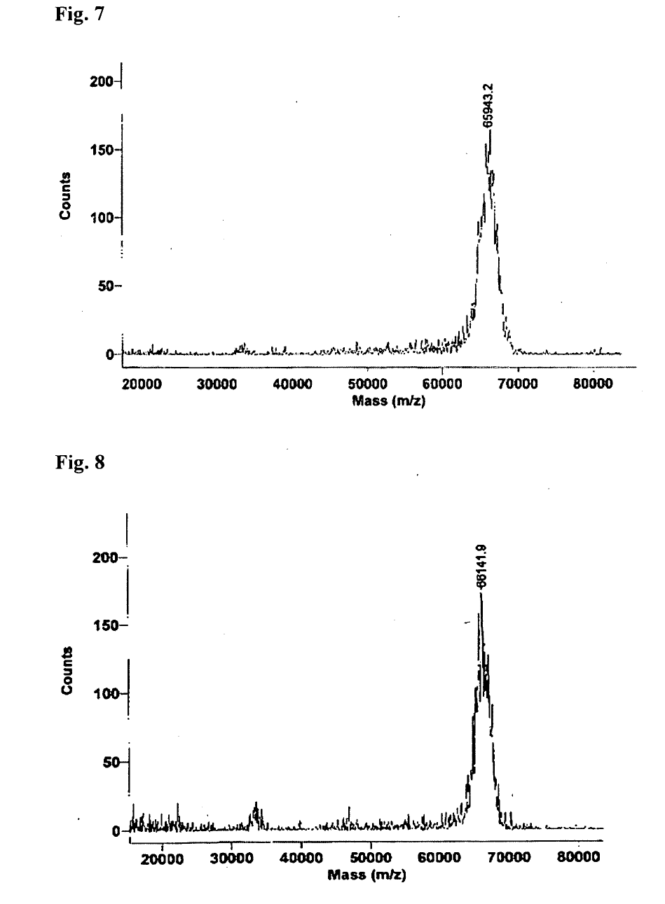

By size-exclusion high performance liquid chromatography, it was confirmed that the mixture of reactants was consisted of about 47% of mono-polyethylene glycol interferon alpha conjugate (mono-PEG-IFN α), about 36% of unmodified interferon α (IFN α), and the others [di-PEGylated interferon alpha conjugate (di-PEG-IFN α) and N-hydroxysuccinimide(NHS)] {see FIG. 1; the absorbance was measured at 280 nm; and the retention time for (a) di-PEG-IFN α was about 8 min, for (b) mono-PEG-IFN α about 9 min, for (c) IFN α about 13.5 min, and for (d)NHS about 15.3 min}. And, the separated mono-three-branched polyethylene glycol interferon alpha conjugated was analyzed by using the MALDI-TOF, as shown in FIG. 7, and the value was 65943.2(m/z) (see FIG. 7).

### Example 2

Preparation of Three-branched Polyethylene Glycol (MW 43,000 Da)-interferon Alpha Conjugate (II) By Using Three-branched Polyethylene Glycol Aldehyde 68 mg of three-branched polyethylene glycol aldehyde (NOF corporation, Japan) having the molecular weight of 43,000 daltons were added to 10 mg of interferon alpha (Dong-A Pharm. Co., Ltd.) in 40 mM of sodium acetate (C₂H₃NaO₂) buffer, pH4.0. The reaction mixture was stirred for 14 hr at cold temperature. And, the reactant was inputted in Hiprep™ 26/10 (Amersham Pharmacia Biotech) desalting column equalized with 40 mM of NaH₂PO₄ (pH4.0) buffer solution, and then the buffer solution was changed by eluting with same buffer solution.

Then, the eluate was inputted in SP-Sepharose Fast Flow cation exchange chromatography (Amersham Pharmacia Biotech) equilibrated with 40 mM of NaH₂PO₄ (pH4.0) buffer solution, and polyethylene glycol interferon alpha conjugate was separated by the liquid chromatography. The polyethylene glycol-interferon alpha conjugate was fractioned using 0˜500mM of concentration gradient of sodium chloride(NaCl).

From the fractioned eluate, interferon alpha remaining after the reaction was excluded through HPLC and SDS-PAGE, to obtain the title conjugate, polyethylen glycol (MW 43,000)-interferon alpha conjugate(II) (mono-three-branched polyethylene glycol interferon alpha conjugate) in which only one three-branched polyethylene glycol was bonded to N-terminus of interferon alpha.

It was confirmed by size-exclusion high performance liquid chromatography that the mixture was consisted of about 42% mono-polyethylene glycol interferon alpha conjugate (mono-PEG-IFN α) and about 55% unmodified interferon α (IFN α) {see FIG. 2; the absorbance was measured at 280 nm, and the retention time of (a) mono-PEG-IFN α was about 9.5 min and that of (b) IFN α was about 14 min}. And, the purity and molecular weight of the separated mono-three-branched polyethylene glycol interferon alpha conjugated were confirmed by using MALDI-TOF, and the value was 66141.9(m/z) (see FIG. 8).

### Comparative Example 1

### Preparation of Two-branched Polyethylene Glycol (PEG, MW 40,000 Da)-interferon Alpha Conjugate(III) Using Two-branched Polyethylene Glycol-N-hydroxysuccinimide

63 mg of two-branched polyethylene glycol N-hydroxysuccinimde (NOF corporation, Japan) having the molecular weight of 40,000 daltons were added to 10 mg of interferon alpha prepared by a known method [Pestka, Sci. Am. 249, 36 (1983)] in 100 mM of bicine buffer, pH8.0. The reaction mixture was stirred for 2 hr at room temperature. And, the reaction was stopped by adding 0.1 M of glycine.

The reactant was inputted into Hiprep™ 26/10 (Amersham Pharmacia Biotech) desalting column equilibrated with 40 mM of NaH₂PO₄ (pH4.0) buffer solution and the buffer solution was changed by eluting with same buffer solution. N-hydroxysuccinimide separated from the three-branched polyethylene glycol-N-hydroxy succinimide by this reaction was removed. An eluant was inputted into SP-Sepharose Fast Flow cation exchange chromatography (Amersham Pharmacia Biotech) equilibrated with 40 mM of NaH₂PO₄ (pH4.0) buffer solution, and polyethylene glycol interferon alpha conjugate was separated therefrom by using the liquid chromatography. The polyethylene glycol-interferon alpha conjugate was fractioned using 0˜500 mM of concentration gradient of sodium chloride(NaCl). The form and size of the fractioned eluate was confirmed by HPLC and SDS-PAGE. And, interferon alpha remaining after the reaction, and interferon alpha conjugates to which two (2) or more two-branched polyethylene glycols are bonded with an interferon alpha, are removed therefrom, to obtain the title conjugate, interferon alpha conjugate to which only one two-branched polyethylene glycol was bonded with an interferon alpha.

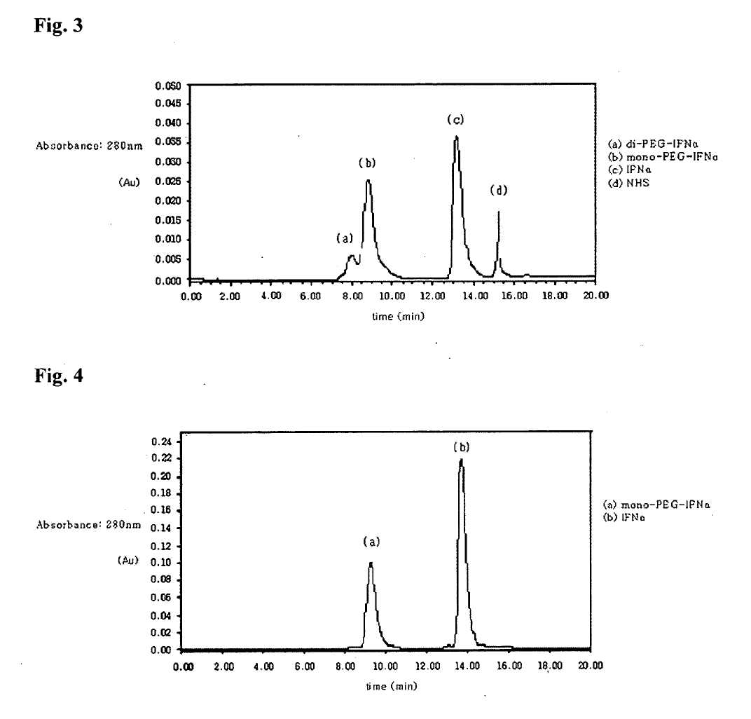

It was confirmed by size-exclusion high performance liquid chromatography that the mixture was consisted of about 40% of mono-polyethylene glycol-interferon alpha conjugate (mono-PEG-IFN CL), about 50% of unmodified interferon α (IFN α), the others [di-PEGylated interferon alpha conjugate (di-PEG-IFN α), and N-hydroxysuccinimide (NHS)] {see FIG. 3; the absorbance was measured at 280 nm, and the retention time of (a) di-PEG-IFN α was about 8 min, that of (b) mono-PEG-IFN CL about 9 min, that of (c) IFN α about 13.5 min, and that of (d)NHS about 15 min}.

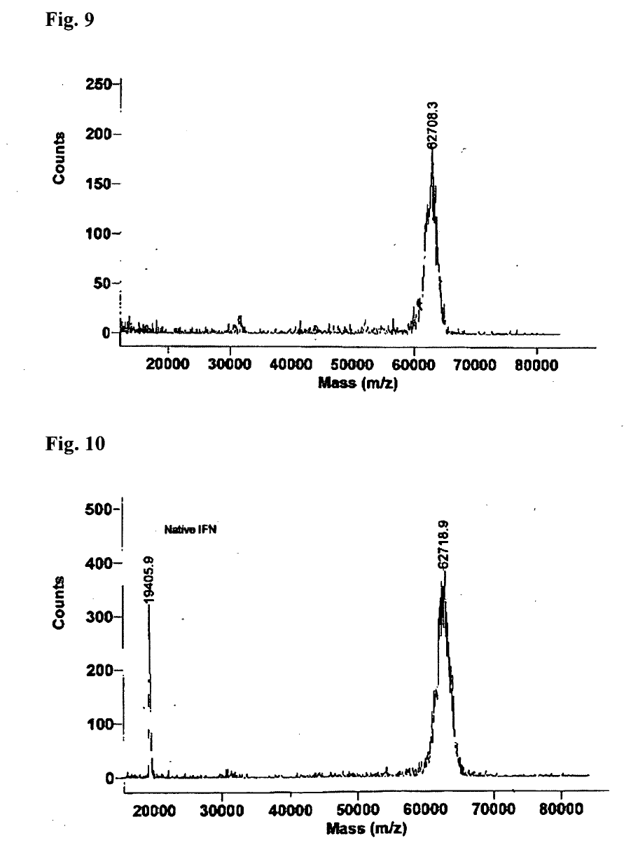

And, the molecular weight was measured for the separated mono-two branched polyethylene glycol interferon alpha conjugated by using MALDI-TOF, and the value was 62708.2(m/z)(see FIG. 9).

### Comparative Example 2

### Preparation of Two-branched Polyethylene Glycol (PEG, MW 40,000 Da)-interferon Alpha Conjugate (IV), By Using Two-branched Polyethylene Glycol Aldehyde

63 mg of two-branched polyethylene glycol aldehyde (NOF corporation, Japan) having the molecular weight of 40,000 daltons were added to 10 mg of interferon alpha (Dong-A Pharm. Co., Ltd.) in 40 mM of sodium acetate (C₂H₃NaO₂) buffer, pH4.0. The reaction mixture was stirred for 10˜14 hr at cold temperature. And, the reactant was inputted in Hiprep™ 26/10 (Amersham Pharmacia Biotech) desalting column equalized with 40 mM of NaH₂PO₄ (pH4.0) buffer solution, and then the buffer solution was changed by eluting with same buffer solution.

Then, the eluate was inputted in SP-Sepharose Fast Flow cation exchange chromatography (Amersham Pharmacia Biotech) equilibrated with 40 mM of NaH₂PO₄ (pH4.0) buffer solution, and polyethylene glycol interferon alpha conjugate was separated by the liquid chromatography. The reactant was fractioned using 0˜500 mM of concentration gradient of sodium chloride(NaCl).

The fractioned eluate was confirmed by HPLC and SDS-PAGE. And, interferon alpha remaining after the reaction was removed therefrom, to obtain polyethylene glycol-interferon alpha conjugate (II) to which only one two-branched polyethylene glycol was bonded with a N-terminal of interferon alpha conjugate.

It was confirmed by size-exclusion high performance liquid chromatography that the mixture was consisted of about 37% of mono-polyethylene glycol interferon alpha conjugate (mono-PEG-IFN α) and about 60% of unmodified interferon α (IFN α) {see FIG. 4; the absorbance was measured at 280 nm, and the retention time of (a) mono-PEG-IFN α was about 9.5 min, and that of (b) IFN α was about 14 min}. And, the molecular weight of the separated mono-three-branched polyethylene glycol-interferon alpha conjugate was measured by using MALDI-TOF, and the value was 62718.9(m/z) (see FIG. 10).

### Comparative Example 3

Preparation of Polyethylene Glycol-interferon Alpha Conjugate Which a Lysine-structured Two-branched Polyethylene Glycol (MW 40,000 Da) Having N-hydroxysuccinimide Ester(NHS ester) Functional Group Was Bonded With an Interferon Alpha

Two-branched polyethylene glycol (MW 40,000) interferon alpha conjugate was prepared by reacting 50 mg of interferon alpha with two-branched polyethylene glycol N-hydroxysuccinimide (Nektar, America, the average molecular weight=40,000 dalton), according to the method described in the Korean Patent No. 10-0254097.

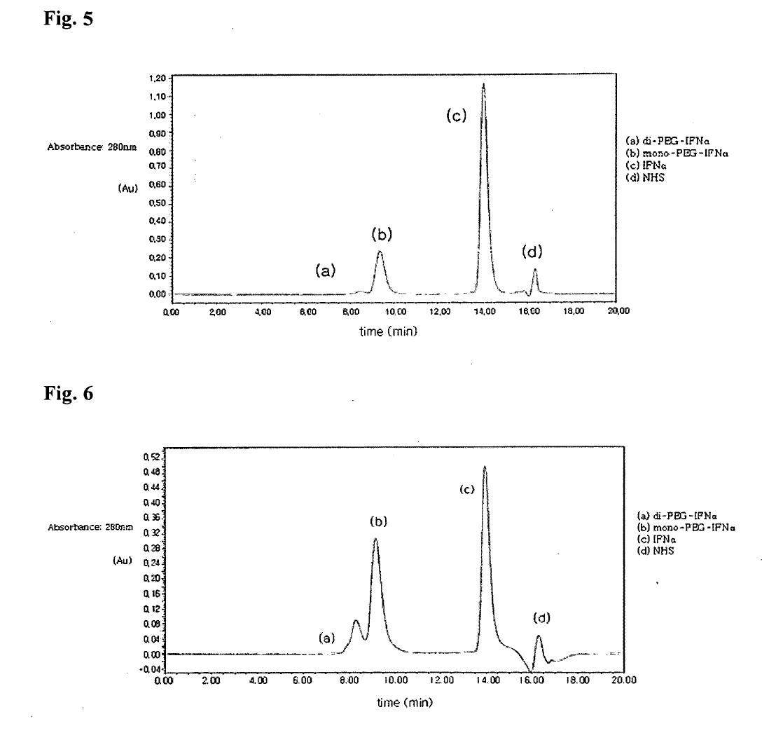

It was confirmed by size-exclusion high performance liquid chromatography that the mixture was consisted of about 17% of mono-polyethylene glycol-interferon alpha conjugate (mono-PEG-IFN α), about 74% of unmodified interferon α (IFN α), and the others [di-PEGylated interferon alpha conjugate (di-PEG-IFN α) and N-hydroxysuccinimide (NHS)] {see FIG. 5; the absorbance was measured at 280 nm, and the retention time of (a) di-PEG-IFN α was about 8.5 min, that of (b) mono-PEG-IFN α about 9.5 min, that of (c) IFN α about 14 min, and that of (d)NHS about 16.5 min}.

### Comparative Example 4

### Preparation of Polyethylene Glycol-interferon Alpha Conjugate Which Lysine-structured Three-branched Polyethylene Glycol (MW 43,000 Da) Having N-hydroxysuccinimide Ester (NHS Ester) Functional Group Was Bonded With an Interferon Alpha

Tri-PEG-NHS (MW 43,000) was prepared by the method described in Korean Patent No. 10-0396983, and then was reacted with 3 mg of interferon alpha to obtain three-branched polyethylene glycol (MW 43,000) interferon alpha conjugate.

It was confirmed by size-exclusion high performance liquid chromatography that the mixture was consisted of about 32% of mono-polyethylene glycol interferon alpha conjugate (mono-PEG-IFN α), about 52% of unmodified interferon c (IFN α), and the others [di-PEGylated interferon alpha conjugate (di-PEG-IFN α) and N-hydroxysuccinimide (NHS)] {see FIG. 6; the absorbance was measured at 280 nm; and the retention time of (a) di-PEG-IFN c was about 8.5 min, that of (b) mono-PEG-IFN c about 9.5 min, that of (c) IFN α about 14 min, and that of (d)NHS about 16.5 min}.

The characterization and pharmacological activity test were conducted by using the conjugates prepared above, and the results are as follows.

### Experimental Example 1

### Reactivity Test of Polyethylene Glycol Derivative and Interferon Alpha

To test the reactivity of polyethylene glycol derivatives and interferon alpha used above, the amount of mono-polyethylene glycol interferon alpha conjugate generated by reacting interferon alpha with polyethylene glycol and the amount of unmodified interferon alpha were determined from peak areas (FIGS. 1 to 6) by size-exclusion high performance liquid chromatography, as shown in the Examples 1 and 2, and the Comparative Examples 1 to 4. As a result, the reactivity of interferon alpha according to the polyethylene glycol structure could be obtained (see Tables 1 and 2).

Considering the remaining amount of unmodified interferon alpha and the amount of generated mono-polyethylene glycol interferon alpha conjugate, the binding reactivity of three-branched polyethylene glycol and interferon alpha in the Examples 1 and 2 were more excellent.

### Experimental Example 2

### The Molecular Weight and Yield of the Polyethylene Glycol Interferon Alpha Conjugate

The polyethylene glycol-interferon alpha conjugates having high purity obtained from the Examples 1 and 2 and Comparative Examples 1 and 2 were analyzed by MALDI-TOF, to confirm that the results correspond to the expected molecular weights (see FIGS. 7, 8, 9 and 10). In relation to the amount of unmodified interferon alpha and the yield of generated mono-polyethylene glycol-interferon alpha conjugate, it was confirmed that Examples 1 and 2 had excellent purified yields[see Table 1(Comparison of the reactivity and yield of polyethylene glycol derivative using N-hydroxysuccinimide and interferon alpha) and Table 2(Comparison of the reactivity and yield of polyethylene glycol deriviative using aldehyde, and interferon alpha)].

TABLE 1

| | Generated mono- | unmodified IFN α | |
|---|---|---|---|
| PEG-IFNα | PEG-IFN α (%) | (%) | Yield (%) |
| Example 1 | 47 | 36 | 23 |
| Comparative | 41 | 50 | 20 |
| Example 1 | | | |
| Comparative | 17 | 74 | 11 |
| Example 3 | | | |
| Comparative | 32 | 52 | 16 |
| Example 4 | | | |

TABLE 2

| | Generated mono- | Unmodified IFN α | |
|---|---|---|---|
| PEG-IFNα | PEG-IFN α (%) | (%) | Yield (%) |
| Example 2 | 42 | 55 | 28 |
| Comparative | 37 | 60 | 24 |
| Example 2 | | | |

### Experimental Example 3

### Antiviral Activity Test and In Vitro Activity Test of Polyethylene Glycol-interferon Alpha Conjugate

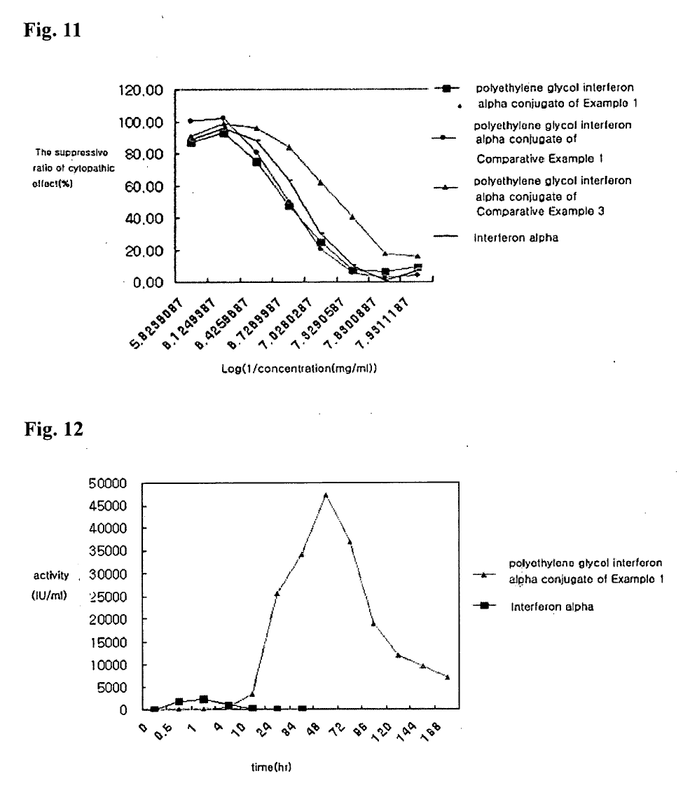

To investigate the effect of the polyethylene glycol derivatives and interferon alpha conjugates used the above with regard to the activity of interferon alpha, the antiviral activities of each of mono-polyethylene glycol interferon alpha conjugates generated in Example 1 and Comparative Examples 1 and 3 were measured by cytopathic effect (CPE) assay using Marbin-Darby Bovine Kidney (MDBK) cells. The cells were challenged with Vesicular Stomatitis Virus(VSV). And, their relative activities to interferon alpha were also measured (see, FIG. 11).

To measure the relative activity, interferon alpha was diluted 10⁵ times, the polyethylene glycol-interferon alpha conjugate of Comparative Example 1 was diluted 2×10⁵ times, the polyethylene glycol-interferon alpha conjugate of Example 1 was diluted 10⁵ times, and the polyethylene glycol-interferon alpha conjugate of Comparative Example 3 was diluted 2×10⁴ times. After 2-folds serial dilute, they were added to Marbin-Darby Bovine Kidney (MDBK) cells and challenged with Vesicular Stomatitis Virus(VSV). After that, continuous dilution ratio values showing TCID₅₀ value (Tissue Culture Infective Dose, 50% of infective dose of tissue culture cells) were calculated, and each activity value was obtained by a statistical method.

The results shown in the following Table 3 suggest that the decrease of biological activity by modification of polyethylene glycol was less in the three-branched polyethylene glycol-interferon alpha conjugate than the two-branched polyethylene glycol-interferon alpha conjugate [see Table 3 (Biological activity of polyethylene glycol-interferon alpha conjugate)].

TABLE 3

| Type | Biological activity (%) |
|---|---|
| IFN a | 100 |
| Example 1 | 4.3 |
| Comparative Example 1 | 3.4 |
| Comparative Example 3 | 3.6 |

### Experimental Example 4

### Pharmacodynamic Test of Polyethylene Glycol-interferon Alpha Conjugate

The pharmacodynamic test was conducted by subcutaneous injection of interferon alpha and polyethylene glycol-interferon alpha conjugate of Example 1 into experimental animals (Sprague Dawley rats) which had 240˜260 g of body weights. After injecting them by the amount of 1×10⁷ IU per head, the blood samples were collected from the rats at 0 min, 30 min, 1 hr, 4 hr, 10 hr, 24 hr, 34 hr, 2 days, 3 days, 4 days, 5 days, 6 days, and 7 days after the injection. The antiviral activities of the samples were measured by the cytophatic effect (CPE) assay, and thus the half-life(T_{1/2}) values of interferon alpha and polyethylene glycol-interferon alpha conjugate were obtained (see FIG. 12).

The half-life in blood of three-branched polyethylene glycol interferon alpha conjugate of Example 1 was increased 9.2 times compared with that of interferon alpha[see, Table 4 (Pharmacodynamics of interferon alpha and polyethylene glycol-interferon alpha in rat (Sprague Dawley rat))]

TABLE 4

| | IFN a | Example 1 |
|---|---|---|
| Cmax (IU/ml) | 2413 | 47214 |
| Tmax (hr) | 1 | 48 |
| MRT (hr) | 5.4 | 87.9 |
| CL/F (ml/hr/kg) | 893.6 | 2.52 |
| Vss/F (ml/kg) | 4803.8 | 221.4 |
| T½ (hr) | 4.5 | 41.5 |
| AUC (IU * hr/ml) | 1.12E+04 | 3.97E+06 |

*The abbreviations in the above table have the following meanings:
Tmax: time to reach the maximum concentration
Cmax: maximum concentration in blood
MRT: Mean Residual Time in blood
CL/F: total plasma clearance
Vss/F: apparent volume of distribution at steady state
t½: elimination half-life
AUC: area under the concentration time curve.

### Experimental Example 5

### Antitumoral Activity Test of Polyethylene Glycol-interferon Alpha Conjugate

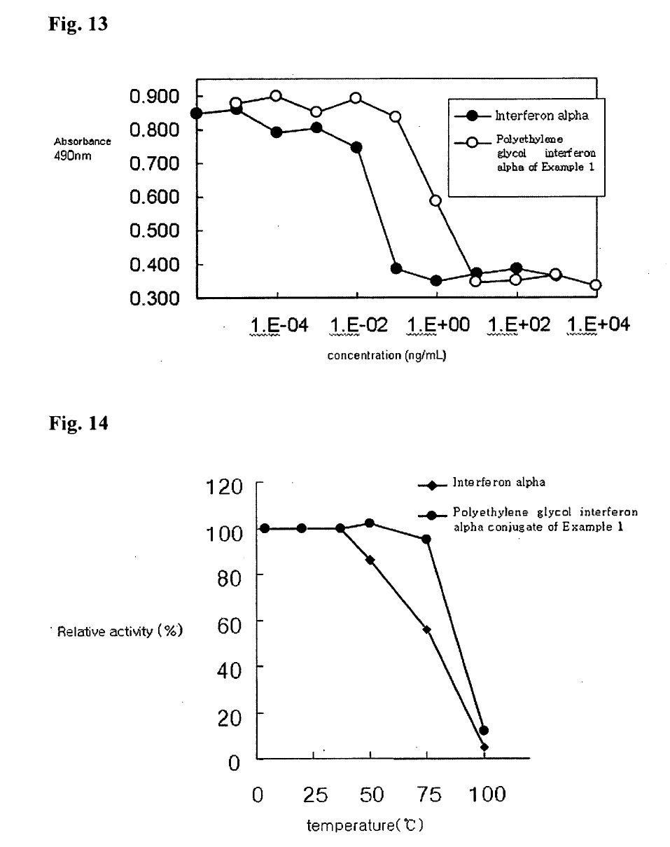

Daudi cells (ATCC CCL-213) were grown in RAPI 1640 (Gibco, America) medium supplemented with 10% fetal bovine serum and penicillin-streptomycin 0.5% at 37° C., CO₂ incubator for 2 days. After the culture was completed, the cell was washed with the medium once, and then diluted to make the density of 10⁶ cell/ml. The interferon alpha and three-branched polyethylene glycol-interferon alpha conjugate of Example 1 were diluted to be 2 mg/ml and 19.2 mg/ml, respectively. And, each of these solutions were serial diluted by 10-folds, to make 10 samples having different concentrations. After that, 100 μ [symbol] of diluents/well of 96-microplate were prepared to all wells except those of the control cells, 50 [symbol] maintenance medium containing VSV was added. As control, the wells containing cells and virus except sample were prepared. The microplate was incubated in CO₂ incubator at 37° C. for 5 days. After 5 days, 40 μ [symbol] of MTS solution comprising PMS (Promega, America) was added to each of the wells, which then were incubated for 1.5 hr. The absorbance was measured for them at 490 nm, to calculate EC₅₀ (50% effective concentration). The results as shown in FIG. 13 suggest that the polyethylene glycol-interferon alpha conjugate has similar antitumoral activity to interferon alpha (see FIG. 13).

### Experimental Example 6

### Temperature Stability Test of Polyethylene Glycol-interferon Alpha Conjugate

Interferon alpha and the three-branched polyethylene glycol interferon alpha conjugate of Example 1 were added to 40 mM NaH₂PO₄ (pH 5.0) of buffer solutions to make 1 mg/ml concentration of solutions respectively. After incubating them at 0° C., 20° C., 37° C., 50° C., 70° C. and 100° C., for 15 min, and cooling down to room temperature, their biological activities were measured(see FIG. 14).

The results in FIG. 14 suggest that the polyethylene glycol-interferon alpha conjugate is pharmaceutically more stable than an unmodified interferon alpha.

### Experimental Example 7

### Stability Test of Polyethylene Glycol Interferon Alpha Conjugate Against a Tryptic Digestion

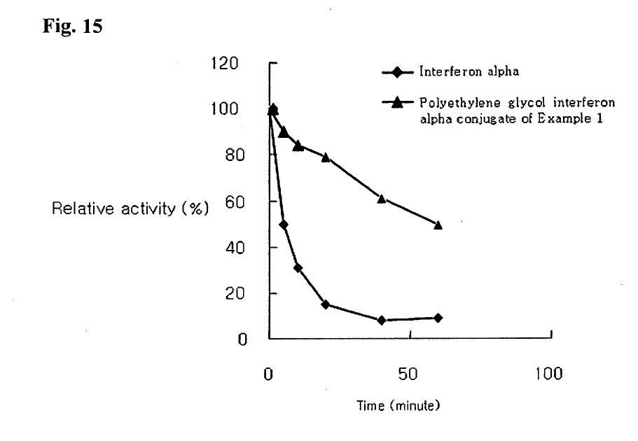

Interferon alpha and the three-branched polyethylene glycol interferon alpha conjugate of Example 1 were prepared to the concentration of 1 mg/ml with buffer solution, and 1 mg of trypsin(pH 7.0) was added per milliliter of solution to induce proteolysis at room temperature, respectively. Aliquots of each solutions were collected at 5 min, 10 min, 20 min, 40 min, and 60 min after starting the reaction, and their biological activities were measured (see FIG. 15).

The results suggest that the polyethylene glycol-interferon alpha conjugate is more stable against protease than an unmodified interferon alpha.

### INDUSTRIAL APPLICABILITY

The present invention relates to biologically active new three-branched polyethylene glycol-interferon alpha conjugates having glycerol structure. So, this invention is characterized in having high purity and high yield, minimizing the reduction of bioactivity, and increasing the half-life in blood, by overcoming the problems that linear polyethylene glycol cannot bond many linear high molecules to protein or peptide; branched high molecular derivatives induce excessive steric hindrance on the surfaces of protein or peptide; and branched high molecular derivatives whose linkers are lengthened have low purification yield by low purity, etc.

Therefore, the pharmaceutical composition of the present invention containing polyethylene glycol-interferon alpha conjugate having antiviral activity and anti-tumoral activity has the effects that the reduction of activity is minimized, and the therapeutic efficiency can be improved, and the patient's compliance can be improved by decreasing the administration frequency due to the lengthened half-life in the body, compared to interferon alpha treatment agent known in the art.

1. Three-branched polyethylene glycol-interferon alpha conjugate of general formula (I), wherein polyethylene glycol has an average molecular weight of from 400 to 45,000 daltons, 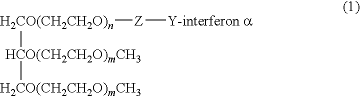
wherein,
n is an integer of 1 to 1,000;
m is an integer of 10 to 1,000;
Z is (CH₂)_{S} or (CH₂)_{S}NHCO(CH₂)_{S} as a linker of interferon alpha and polyethylene glycol wherein S is an integer of 1 to 6;
Y is a secondary amine or an amide bond which is a bond of NH₂ functional group in interferon molecule, and a functional group of polyethylene glycol derivative

2. The conjugate of claim 1, wherein the polyethylene glycol has an average molecular weight of from 30,000 to 45,000 daltons.

3. The conjugate of claim 1, wherein the polyethylene glycol has the average molecular weight of 43,000 daltons.

4. A method of preparing three-branched polyethylene glycol-interferon alpha conjugate of general formula (1), wherein polyethylene glycol has an average molecular weight of from 400 to 45,000 daltons, comprising forming a covalent bond of branched polyethylene glycol derivative of general formula (2) and interferon alpha. 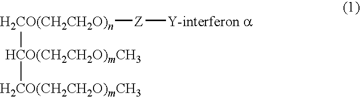
wherein,
n is an integer of 1 to 1,000;
m is an integer of 10 to 1,000;
Z is (CH₂)_{S} or (CH₂)_{S}NHCO(CH₂)_{S} as a linker of interferon alpha and polyethylene glycol wherein S is an integer of 1 to 6;
Y is a secondary amine or an amide bond which is a bond of NH₂ functional group of interferon molecule and a functional group of polyethylene glycol derivative; 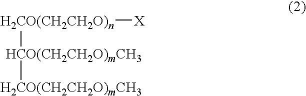
wherein,
n is an integer of 1 to 1,000; m is an integer of 10 to 1,000; X is a functional group represented by the following general formula (3) which can react chemically with protein or peptide containing interferon alpha 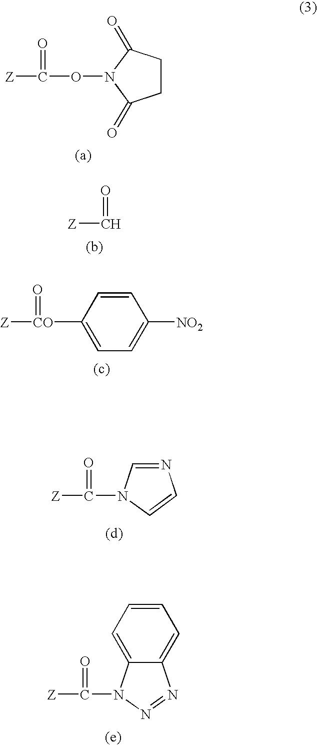
Z is (CH₂)_{S} or (CH₂)_{S}NHCO(CH₂)_{S} as a linker of interferon alpha and polyethylene glycol wherein S is an integer of 1 to 6.

5. The method of claim 4, wherein the polyethylene glycol has an average molecular weight of from 30,000 to 45,000 daltons.

6. The method of claim 4, wherein the polyethylene glycol has the average molecular weight of 43,000 daltons.

7. The method of claim 4, wherein X is (a) or (b) in the general formula (3).

8. The method of claim 4, wherein the molar ratio of interferon alpha to three-branched polyethylene glycol derivative is from 1:0.5 to 1:50.

9. The method of claim 8, wherein the molar ratio of interferon alpha to three-branched polyethylene glycol derivative in the reaction is from 1:0.5 to 1:3.

10. A pharmaceutical composition for treating or preventing interferon alpha receptive diseases comprising the conjugate of claim 1, in an amount effective for treating or preventing interferon alpha receptive diseases.

11. The composition of claim 10, wherein the interferon alpha receptive diseases are selected from the group consisting of hairy cell leukemia, Kaposi's sarcoma, chronic Myelogenous Leukemia (CML), B-cell lymphoma, T-cell lymphoma, melanoma, myeloma, and renal cell carcinoma.

12. A method of treating or preventing interferon alpha receptive diseases comprising administering to a subject in need thereof the conjugate of claim 1, in an amount effective for treating or preventing interferon alpha receptive diseases.

13. A pharmaceutical composition for treating or preventing interferon alpha receptive diseases comprising the conjugate of claim 2 in an amount effective for treating or preventing interferon alpha receptive diseases.

14. A pharmaceutical composition for treating or preventing interferon alpha receptive diseases comprising the conjugate of claim 3 in an amount effective for treating or preventing interferon alpha receptive diseases.

15. A method of treating or preventing interferon alpha receptive diseases comprising administering to a subject in need thereof the conjugate of claim 2 in an amount effective for treating or preventing interferon alpha receptive diseases.

16. A method of treating or preventing interferon alpha receptive diseases comprising administering to a subject in need thereof the conjugate of claim 3 in an amount effective for treating or preventing interferon alpha receptive diseases.

17. The method of claim 12, wherein the interferon alpha receptive diseases are selected from the group consisting of hairy cell leukemia, Kaposi's sarcoma, chronic Myelogenous Leukemia (CML), B-cell lymphoma, T-cell lymphoma, melanoma, myeloma, and renal cell carcinoma.
